(19) United States
(12) Patent Application Publication
(10) Pub. No.: US 2024/0032899 A1
(43) Pub. Date: Feb. 1, 2024

(54) DISPOSABLE SALIVA COLLECTOR FOR WIND INSTRUMENTS

(71) Applicant: Mihir Mahesh, Irving, TX (US)

(72) Inventor: Mihir Mahesh, Irving, TX (US)

(21) Appl. No.: 18/136,008

(22) Filed: Apr. 18, 2023

Related U.S. Application Data
(60) Provisional application No. 63/392,144, filed on Jul. 26, 2022.

(51) Int. Cl. A61B 10/00

(52) U.S. Cl. CPC A61B 10/0051; A61B 10/0096

(57) ABSTRACT

The present invention provides a disposable saliva collector for a wind instrument, comprising polygonal soft and flexible material bounded by straight or curved sides; wherein, the polygonal soft and flexible material is bound by straight or curved sides encompassing an absorbent material and leak guards on sides of the absorbent material; an anchoring hole on the opposite axis of the leak guard; and fastening straps on the same axis as the leak guards.

### FIELD OF THE INVENTION

The present invention relates to a disposable saliva collector for wind instruments, specifically providing a hygienic and convenient way to collect discharged saliva from a wide variety of wind instruments. This disposable saliva collector is designed as a reversible attachment.

### BACKGROUND OF THE INVENTION

Musical wind instruments, such as trombones, trumpets, and others, collect human saliva during use. Typically, wind instruments are designed with a specific part that allows saliva to accumulate and a valve or tap, commonly known as a water key, that can be used to drain the accumulated saliva. Musicians can use the water key to drain saliva at any time and location. Because of this, it is common for musicians to discard the drained saliva on the floor, even during performances or practice locations.

The aforementioned practice of discarding saliva on the floor is unhygienic. This practice has been accepted as a norm for many decades. However, with the advent of the pandemic and heightened awareness of the importance of hygiene, it has become necessary to address this situation and devise a better method for collecting, draining, and disposing of saliva from wind instruments.

Numerous inventors in the past have attempted to address the unhygienic practice of discarding saliva from wind instruments. These inventors have created various products, which hereinafter will be referred to as “Saliva Accumulator(s),” that collect saliva for later disposal. However, these inventions have not been widely adopted due to their poor convenience and usability. As a result, the unhygienic practice of discarding saliva on the floor continues to be prevalent in the music industry.

A patent application, identified as JP2022051348A, discloses a specific Saliva Accumulator design. This collector is designed to collect accumulated saliva in aerophones that have a saliva discharge port, which is typically opened or closed using a water key. The collector includes a receiving part with an opening for collecting saliva and a storage part for storing the saliva. The receiving part is tapered towards the storage part, with a through-hole located at the leading end to enable connection between the receiving part and storage part.

US2020320963 provides a Saliva Accumulator which comprises of container that provides a space to accommodate at least one absorber, and a head wherein one end is connected to at least one part of a musical instrument and another end can join with the first end so the absorber will stick to the instrument tightly without falling off.

JP2004151615 provides a Saliva Accumulator that can securely prevent the dropping of saliva with a simple structure, which hygienically collects saliva discharged from a wind instrument and does not spoil the performance atmosphere. The saliva collector for collecting saliva is detachably applied to the discharge part for saliva. The saliva collector is formed of a water-absorbing body composed of nonwoven cloth, water-absorbing paper, high-absorption polymer, or the like, having a high-water absorbing property; and

U.S. Pat. No. 4,016,794 is a Saliva Accumulator that provides a cup-like device designed to attach to various musical instruments for collecting and retaining saliva emitted from the water key during the extended playing of such instruments.

Saliva Accumulators have been found to have poor convenience and usability due to certain drawbacks. Typically, these accumulators collect liquid saliva in a container that is attached to the wind instrument. The collected saliva needs to be emptied and the container cleaned out at a later time. Until it is cleaned out, the saliva remains in the container, and most musicians find it unpleasant to carry around collected liquid saliva.

Furthermore, cleaning and sterilizing the containers are additional undesirable activities for most musicians. Some Saliva Accumulators are not designed to be used on wind instruments with a water key.

The above-mentioned issue is further exacerbated by the fact that musicians may prefer to use multiple wind instruments during a performance or practice session, each of which may have a slightly different design and dimensions. Typically designs of Saliva Accumulators are rigid. They are tailored to be affixed to the wind instruments in a certain way. However, wind instruments, which are manufactured by different companies, have minor variations in their designs and dimensions. A less than perfect fit may cause undesirable leakage. This means that a musician would need to have multiple saliva collectors, each custom designed to fit the specific wind instrument. The added expense and inconvenience of having to obtain and carry multiple collectors can be a significant deterrent to the adoption of such products.

### SUMMARY OF THE INVENTION

The present invention aims to overcome the drawbacks of prior art by providing a disposable saliva collector as a reversible attachment, which can collect discharged saliva from a wide range of wind instruments in a convenient and hygienic manner.

Another object of the invention is to provide a disposable saliva collector attachment for wind instruments that are lightweight, easy to carry, and fabricated economically.

Yet another object of the invention is to provide a disposable saliva collector attachment for wind instruments that is easy to install, compact, and inexpensive.

Yet another object of the invention is to provide a disposable saliva collector attachment for wind instruments that does not require any special training, tools, or customization for attaching and removing from a wind instrument.

In an embodiment of the present invention, the invention provides a disposable saliva collector for wind instrument comprising polygonal soft and flexible material bounded by straight or curved sides; wherein, the polygonal soft and flexible material is bound by straight or curved sides encompassing an absorbent material and leak guards on sides of the absorbent material; an anchoring hole on the opposite axis of the leak guard; and fastening straps on the same axis as the leak guards.

In an embodiment of the present invention, the polygonal soft and flexible material of the disposable saliva collector is a soft and flexible material made up of composite fiber, cloth, or paper, and combinations thereof, and the absorbent material is placed on the side of the absorbent material with at least one anchoring hole is provided on the opposite axis of the leak guard. Further, the disposable saliva collector has anchoring holes provided preferably at the top which can be inserted into the water key of the wind instrument.

In another embodiment of the present invention, leak guards of the disposable saliva collector prevent the liquid from leaking at the sides, and the adhesive or hook and loop fastener securely fixes the disposable saliva collector to the wind instrument.

In another embodiment of the present invention, the invention provides a distinct technical advantage over the state-of-the-art devices wherein the present invention addresses the issue of a correct fit and retro fit for wind instruments whose designs and dimensions vary due to varying diameter of the instruments, varying alignment of the water key lever and varying shapes of the hinge and saliva collection area of the wind instruments. This fitting is achieved by designing a flexible product that can accommodate varying instrument dimensions and compensates for the varying diameter of the instruments, varying alignment of the water key lever, and varying shapes of the hinge and saliva collection area of the wind instruments. The design of the water key hinge and saliva collection and emptying area vary quite remarkably between different wind instruments. This poses an additional challenge to design the products.

In an embodiment of the present invention, the disposable saliva collector provides a specific advantage wherein the disposable saliva collector allows the water key lever to be operated as usual, without restricting its normal operation, allowing its full range of operation. When the water key lever is operated to open, the collected saliva flows out of the musical instrument as usual, but instead of falling onto the floor, it is collected in the disposable saliva collector. The disposable saliva collector has minimal or no effect on the quality of sound from the musical instrument.

In another embodiment, the disposable collector is disposed of by unwrapping it. Alternatively, the absorbent material is disposed of and replaced by an unused or new absorbent material.

In another embodiment, the invention provides a method of attaching disposable saliva collector to a wind instrument having a water key lever attached at an angle to the windpipe, the method comprising steps of:
placing a disposable saliva collector in communication with the water key lever with absorbent material facing towards the water keyhole;
pulling the first anchor hole over the water key lever to fix its position around the wind instrument's windpipe;
pulling the second anchor hole over the water key lever to form a complete wrap around the instrument windpipe; and
securing the disposable saliva collector using the provided elastic fastening straps.

In another embodiment, the invention provides a method of attaching a disposable saliva collector to a wind instrument having a water key lever attached straight to the windpipe, the method comprises steps of
placing the disposable saliva collector in communication with the water key lever with absorbent material facing towards the water keyhole;
pulling the anchoring hole over the water key lever to fix its position around the instrument windpipe;
wrapping the disposable saliva collector around the instrument windpipe; and
securing the disposable saliva collector using the provided elastic fastening straps.

### BRIEF DESCRIPTION OF THE DRAWING

The object of the invention may be understood in more detail and a more particular description of the invention is briefly summarized above by reference to certain embodiments thereof which are illustrated in the appended drawings, which drawings form a part of this specification. It is to be noted, however, that the appended drawings illustrate preferred embodiments of the invention and are therefore not to be considered limiting of its scope, for the invention may admit to other equally effective equivalent embodiments.

FIG. 1 illustrates the front view of the disposable saliva collector attachment according to an embodiment of the present invention;

FIG. 2 illustrates the back view of the disposable saliva collector attachment according to an embodiment of the present invention;

FIG. 3 illustrates the front view of another disposable saliva collector according to an embodiment of the present invention;

FIG. 4 illustrates the back view of another disposable saliva collector according to an embodiment of the present invention;

FIG. 5 illustrates the assembly of a disposable saliva collector for a wind instrument having a water key lever attached at an angle to the windpipe; and

FIG. 6 illustrates the assembly of a disposable saliva collector for a wind instrument having a water key lever attached straight to the windpipe.

### DETAILED DESCRIPTION OF THE INVENTION

The present invention will now be described more fully hereinafter with reference to the accompanying drawings in which a preferred embodiment of the invention is shown. This invention may, however, be embodied in many different forms and should not be construed as being limited to the embodiment set forth herein. Rather, the embodiment is provided so that this disclosure will be thorough, and will fully convey the scope of the invention to those skilled in the art.

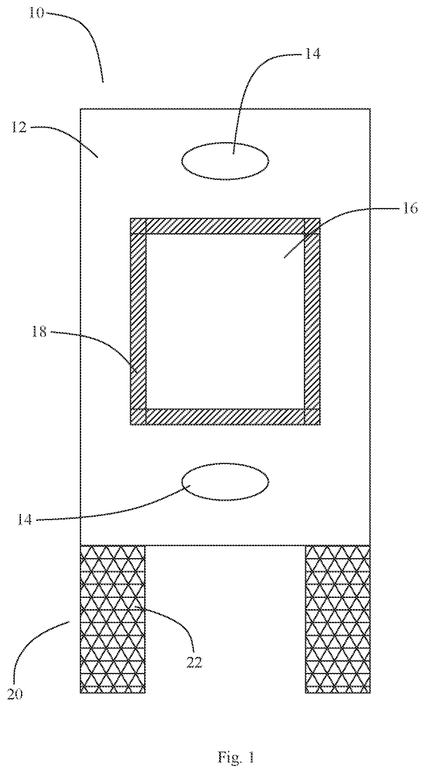

Referring to FIG. 1 shows a front view of a disposable saliva collector 10 for the wind instrument attached to the water key lever at an angle to the windpipe. The disposable saliva collector 10 is made up of a polygonal soft and flexible material 12 including but not limited to composite fiber, cloth, or paper, and combinations thereof. Further, the polygonal soft and flexible material 12 has a plurality of anchoring holes 14 provided primarily at the top and bottom for affixing to the water key of the wind instrument. Further, in the center of the disposable saliva collector 10, an absorbent material 16 is provided for absorbing the discarded saliva through the water key of the wind instrument. Furthermore, the edges of the absorbent material 16 have leak guards 18 that prevent the liquid from leaking at the sides. The absorbent material 16 is composed of materials such as nonwoven cloth, water-absorbing paper, high-absorption polymer, or the like, that can absorb liquids and provide means for hygienically handling the disposal of saliva. Towards the bottom, the disposable saliva collector 10 has a plurality of elastic fastening straps 20 provided with adhesive or hook and loop fastener 22 to securely fix the disposable saliva collector to any wind instrument.

Further, in an embodiment of the present invention, the back of the disposable saliva collector 10 optionally has an insertion window for easily slipping in the absorbent material 16. This facilitates musicians who prefer disposing of and replacing just the absorbent material 16 instead of disposing of and replacing the entire cover 10.

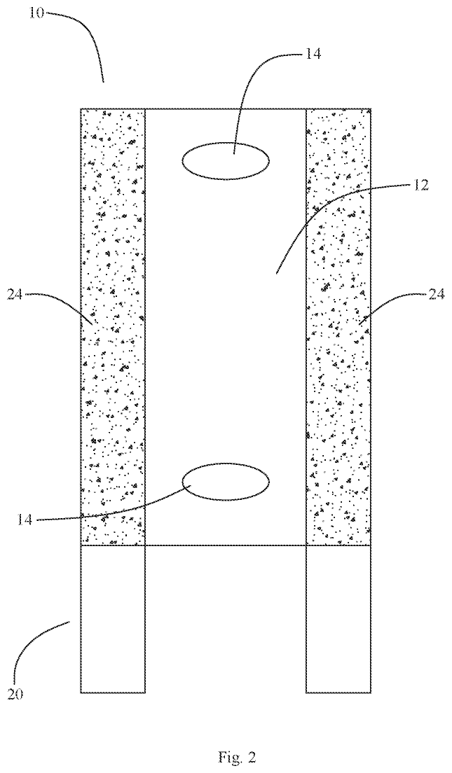

Referring to FIG. 2 shows a back view of a disposable saliva collector 10 having the water key lever at an angle to the windpipe of the wind instrument. The back of the polygonal soft and flexible material 12 has a plurality of adhesive or hook and loop fasteners 24 to fix the disposable saliva collector to any wind instrument securely.

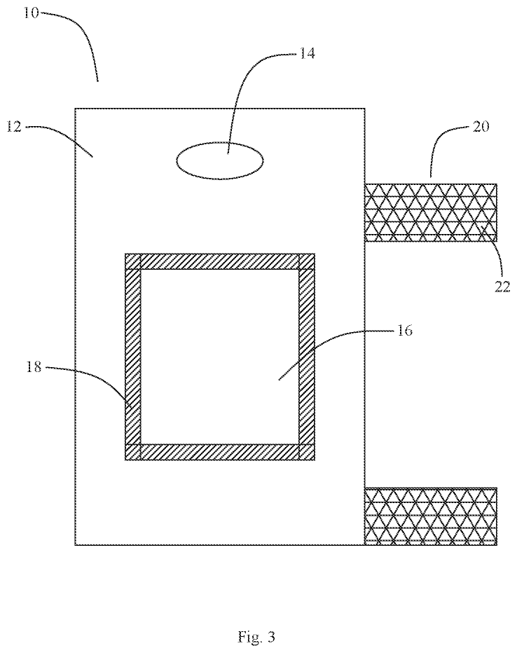

Referring to FIG. 3 shows the front view of a disposable saliva collector 10 for a wind instrument that has the water key lever attached inline to the windpipe. The disposable saliva collector 10 is made up of polygonal soft and flexible material 12 including but not limited to composite fiber, cloth, or paper, and combinations thereof. Further, the polygonal soft and flexible material 12 has anchoring holes 14 provided preferably at the top which is inserted into the water key of the wind instrument. Towards the center of the disposable saliva collector 10, an absorbent material 16 is placed for absorbing the discarded saliva through the water key of the wind instrument, and around the edges of the absorbent material 16 has leak guards 18 that prevent the liquid from leaking at the sides. The absorbent material 16 is composed of materials such as nonwoven cloth, water-absorbing paper, high-absorption polymer, or materials alike, that absorb liquids and provide means for hygienically handling the disposal of saliva. Towards the bottom, the disposable saliva collector 10 has a plurality of elastic fastening straps 20 provided with adhesive or hook and loop fastener 22 to securely fix the disposable saliva collector to any wind instrument.

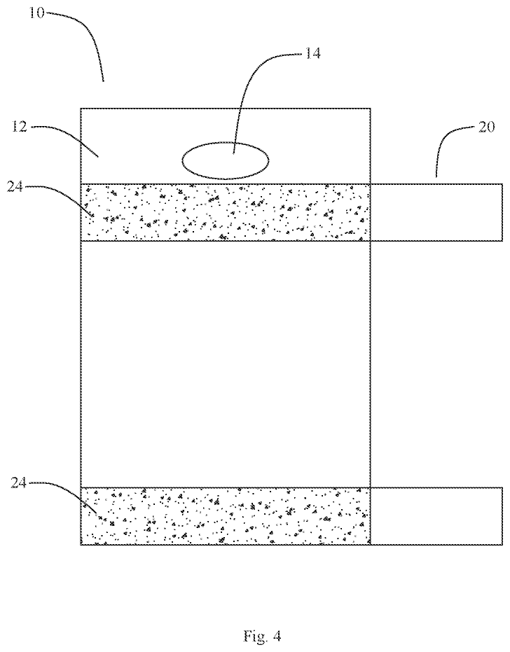

Referring to FIG. 4 shows the back view of a disposable saliva collector 10 for the wind instrument having the water key lever 40 attached straight to the windpipe 30. In the back, the polygonal soft and flexible material 12 has a plurality of adhesive or hook and loop fasteners 24 to fix the disposable saliva collector to any wind instrument securely.

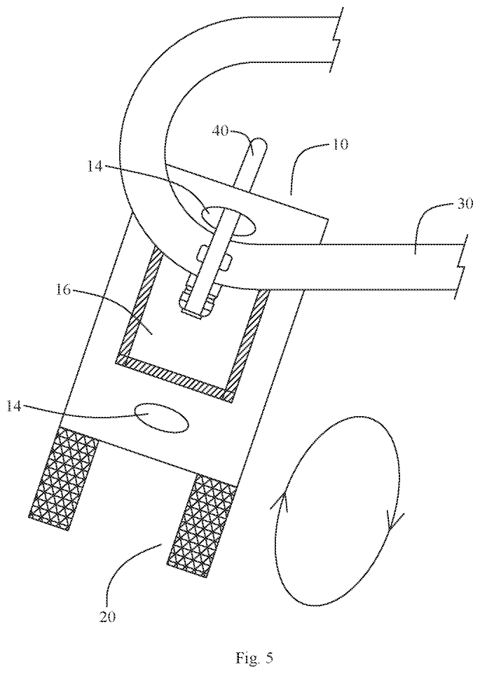

Referring to FIG. 5 shows the assembly of a disposable saliva collector 10 for a wind instrument having a water key lever 40 attached at an angle to the windpipe 30. First, the disposable saliva collector 10 is placed under the water key lever 40 with absorbent material 16 facing toward the water keyhole. Then the top anchor hole 14 is pulled over the water key lever 40 to be affixed around the instrument windpipe 30. Now, the disposable saliva collector 10 can be rolled around the windpipe 30 and the second anchor hole 14 will be pulled over the water key lever 40 to form a complete wrap. Finally, to secure the disposable saliva collector 10 over the windpipe 30, elastic fastening straps 20 are fastened together, and the disposable saliva collector 10 will provide means for hygienically handling the disposal of saliva.

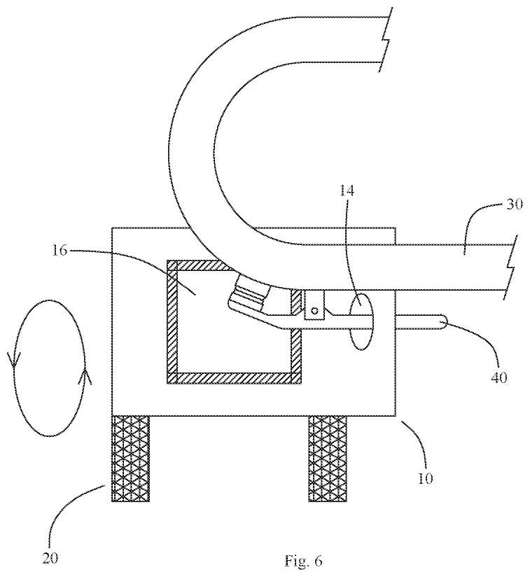

Referring to FIG. 6 is showing the assembly of a disposable saliva collector 10 for wind instruments having a water key lever 40 attached straight to the windpipe 30. First, the disposable saliva collector 10 is placed under the water key lever 40 with absorbent material 16 facing toward the water keyhole. Then the anchor hole 14 is pulled over the water key lever 40 to affix its position around the instrument windpipe 30 and to form a complete wrap. Finally, to secure the disposable saliva collector 10 over the windpipe 30, elastic fasteners 20 are fastened together, and the disposable saliva collector 10 has means for hygienically handling the disposal of saliva.

Through the above description, it is well established that the disposable saliva collector 10 does not require any special training or tools for attaching and removing from the instrument and can hygienically collect the discharged saliva from a wide variety of wind instruments. Further, the disposable saliva collector 10 is easy to install, compact, and inexpensive. In addition, the disposable saliva collector 10 is lightweight, easy to carry, and can be fabricated economically.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. To maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many applications. Any of the described structures and components could be disposable or reusable as desired for a particular use environment. Any component could be provided with a user-perceptible marking to indicate material, configuration, at least one dimension, or the like pertaining to that component, the user-perceptible marking potentially aiding a user in selecting one component from an array of similar components for a particular use environment. A “predetermined” status may be determined at any time before the structures being manipulated reach that status, the “predetermination” being made as late as immediately before the structure achieves the predetermined status. The term “substantially” is used herein to indicate a quality that is largely, but not necessarily wholly, that which is specified a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations.

I claim:

1. A disposable saliva collector 10 for wind instruments, comprising polygonal soft and flexible material 12 bounded by straight or curved sides placed under the water key lever; wherein, polygonal soft and flexible material 12 is encompassing an absorbent material 16 and leak guards 18 placed on the sides of the absorbent material 16; an anchoring hole 14 on the opposite axis of the leak guard 18; and elastic fastening straps and hook and loop fasteners 24 on the same axis as the leak guards 18 allowing the disposable saliva collector 10 to be fastened to the wind instruments.

2. The disposable saliva collector 10 as claimed in claim 1, wherein the polygonal soft and flexible material 12 is a rectangular soft and flexible material made up of composite fiber, cloth, or paper, and combinations thereof and the disposable saliva collector 10 is placed under the water key lever.

3. The disposable saliva collector 10 as claimed in claim 1, wherein the absorbent material is on both sides of the collector 16.

4. The disposable saliva collector 10 as claimed in claim 1, wherein at least one anchoring hole 14 is provided on the opposite axis of the leak guard 18.

5. The disposable saliva collector 10 as claimed in claim 1, wherein has anchoring holes 14, provided at opposite ends of the length of the apparatus, that can be inserted into the water key of the wind instrument.

6. The disposable saliva collector 10 as claimed in claim 1, wherein leak guards 18 prevent the liquid from leaking at the sides.

7. The disposable saliva collector 10 as claimed in claim 1, wherein the adhesive or hook and loop fastener 22 conveniently reversibly secures the disposable saliva collector to the wind instrument while allowing the water key lever to be operated without restricting its normal operation, allowing its full range of operation with minimal or no effect on the quality of sound from the musical instrument.

8. The disposable saliva collector 10 as claimed in claim 1, wherein the wind instrument can be any wind instrument such as Trombone, Trumpet, etc.

9. A method of attaching disposable saliva collector 10 to a wind instrument having a water key lever attached at an angle to the windpipe, the method comprising steps of placing a disposable saliva collector 10 in communication with the water key lever with absorbent material 16 facing towards the water keyhole;
pulling the first anchor hole 14 over the water key lever to fix its position around the wind instrument's windpipe;
pulling the second anchor hole 14 over the water key lever to form a complete wrap around the instrument windpipe; and
securing the disposable saliva collector 10 using the provided elastic fastening straps 20.

10. A method of placing a disposable saliva collector 10 to a wind instrument having a water key lever attached straight to the windpipe, the method comprises steps of:
placing the disposable saliva collector 10 in communication with the water key lever with absorbent material facing towards the water keyhole;
pulling the anchoring hole 14 over the water key lever to fix its position around the instrument windpipe;
wrapping the disposable saliva collector 10 around the instrument windpipe; and
securing the disposable saliva collector using the provided elastic fastening straps 20.
